SEQUENCE LISTING (6 sequences)

SEQ ID NO: 1 - DNA, 20 bp, synthetic construct
  source 1..20: mol_type = other DNA
tagccagtgacaggacccag

SEQ ID NO: 2 - DNA, 23 bp, synthetic construct
  source 1..23: mol_type = other DNA
cagctcaagtcctagttggcaaa

SEQ ID NO: 3 - DNA, 21 bp, synthetic construct
  source 1..21: mol_type = other DNA
cgccaaacccaatgtgatcct

SEQ ID NO: 4 - DNA, 21 bp, synthetic construct
  source 1..21: mol_type = other DNA
ttggacacttcggtcaacaga

SEQ ID NO: 5 - DNA, 21 bp, synthetic construct
  source 1..21: mol_type = other DNA
atgaccacagtccatgccatc

SEQ ID NO: 6 - DNA, 24 bp, synthetic construct
  source 1..24: mol_type = other DNA
ccagtggatgcagggatgatgttc

(19) United States
(12) Patent Application Publication
(10) Pub. No.: US 2025/0381166 A1
(43) Pub. Date: Dec. 18, 2025

(54) Cannabis-Derived Flavonoids And Related Methods

(71) Applicant: CANURTA INC., Mississauga (CA)

(72) Inventors: Jasmin LALONDE, Guelph (CA); Begum ALURAL, Guelph (CA); Alicyia Helena WALCZYK-MOORADALLY, Guelph (CA); Jennifer HOLBORN, Guelph (CA)

(21) Appl. No.: 18/834,584

(22) PCT Filed: Nov. 8, 2022
(86) PCT No.: PCT/CA2022/051648

§ 371 Date: Jul. 30, 2024

Related U.S. Application Data
(60) Provisional application No. 63/304,699, filed on Jan. 31, 2022.

(51) Int. Cl. A61K 31/352; A61K 31/7048; A61K 36/185; A61K 45/06; A61P 35/00

(52) U.S. Cl. CPC A61K 31/352; A61K 31/7048; A61K 36/3482; A61K 45/06; A61P 35/00

(57) ABSTRACT

Provided herein, is a method for cancer therapy through the administration of pharmaceutical compositions comprising cannabis-specific flavonoids such as cannflavins. The flavonoids provided herein, inhibit Trk inhibitory activity therefore can be therapeutically effective for treating RTK/Trk-associated cancers. Also provided is a pharmaceutical or natural health product comprising cannflavin A and/or cannflavin B and/or cannflavin C for treating and/or preventing cancer. Such cancers are brain cancer, breast cancer, colorectal cancer, prostate cancer, pancreas cancer, ovarian cancer, lung cancer, bladder cancer, melanoma, thyroid cancer, head and neck cancers, uterine sarcoma, and/or neuroblastoma adrenocortical carcinoma.

### RELATED APPLICATIONS AND INCORPORATION BY REFERENCE

This application is the national phase entry of international application no. PCT/CA2022/051648 filed Nov. 8, 2022, which claims the benefit of U.S. provisional application No. 63/304,699, filed Jan. 31, 2022, which is herein incorporated by reference in its entirety for all purposes.

### REFERENCE TO A SEQUENCE LISTING SUBMITTED AS AN XML FILE

The Sequence Listing written in file 617846SEQLIST.xml is 8 kilobytes, was created on Jul. 22, 2025, and is hereby incorporated by reference in its entirety.

### FIELD

Described herein are cannabis-derived flavonoids or cannflavins. More specifically, described herein are compositions and methods comprising cannflavins for inhibiting RTKs.

### BACKGROUND

Flavonoids are polyphenolic compounds found in various plant-derived foods and beverages.

Apart from the psychoactive molecule A9-tetrahydrocannabinol (THC) and other related cannabinoids with only mild or no psychotropic effect, like cannabidiol (CBD) and cannabigerol (CBG), the Cannabis sativa plant also produces hundreds of secondary metabolites including at least twenty different flavonoid compounds (Flores-Sanchez et al., 2008). Among those, the flavones cannflavin A and cannflavin B are considered to accumulate uniquely in C. sativa cultivars. Seminal work by Barrett and colleagues performed more than 30 years ago helped identify these two flavonoids and characterize them as inhibitors of prostaglandin E2 production with the ability to produce anti-inflammatory effects that are thirty times more potent than aspirin (Barrett et al., 1985; 1986). However, a broader understanding of cannflavins' influence on cell biology in health and disease did not progress much since their initial description because of challenges associated with their extraction and limited distribution. Although some findings provide novel insights about the pharmacological potential of cannflavins or related molecules (Eggers et al., 2019; Moreau et al., 2019), the full range of molecular changes induced by cannflavins in cells remains to be described.

Cellular processes such as cell proliferation, differentiation, cell invasion and mobility, and apoptosis are often controlled by protein kinases (PKs), and lack of PK regulation is frequently associated to the development of many disorders, including cancers. Accordingly, since PKs are often seen to play important roles during various stages of tumor development, they constitute essential pharmaceutical targets for cancer treatments. One class of PK is formed by receptor tyrosine kinases (RTKs) which are high-affinity cell surface receptors (e.g., EGFR, PDGFR, VEGFR, IGFR, FGFR, TRK, AXL, RET) for many polypeptide growth factors, cytokines, and hormones (Barbacid et al., 1991). One particular group of receptor tyrosine kinases is comprised of the tropomyosin-related kinase (Trk) family members TrkA, TrkB, and TrkC. These RTKs are regulated by neurotrophins, a class of secreted growth factors responsible for the development and function of neurons, hence the activation of these receptors has significant effects on the functional properties of neurons.

The first Trk was identified as an oncogenic fusion alteration (Pulciani et al., 1982). Since then, other genetic alterations have been identified in TrkA, TrkB, and TrkC, and deregulation of these specific RTK proteins and their ligands has been described in various types of tumors, including colon, prostate, pancreas, ovarian, lung, bladder, breast, melanoma, thyroid, head and neck cancers, as well as neuroblastoma (Solomon et al., 2019). As a result, the interest in this family of receptors has been revived, and inhibitors of Trks are being explored as potential treatment options for cancer treatments (Cocco et al., 2018; Lange and Lo, 2018; Wang et al., 2020).

Unlike TrkA and TrkC, the primary mechanism of TrkB activation in human tumors seems to be through overexpression of the full-length protein (Geiger and Peeper, 2005). Several more recent studies have shown that TrkB and its primary ligand, brain-derived neurotrophic factor (BDNF), play a role in cancer development and metastasis, and are associated with poor survival in patients with various cancer types. Notably, aberrant BDNF/TrkB signaling was found activated in breast (Kin et al., 2016), colon (Shen et al., 2019), lung (Sinkevicius et al., 2014), pancreatic (Miknyoczki et al., 1999), and ovarian cancers (Xu et al., 2019), cutaneous melanoma (Antunes et al., 2019) [22], and oral squamous cell carcinoma (OSCC) (de Moraes et al., 2019). Abnormal neurotrophin signaling via TrkB is also seen as an important factor in various types of brain tumours, including glioblastomas. Glioblastoma multiforme (GBM) is the most common and aggressive type of adult brain cancer. These tumours can occur in any region of the central nervous system and the average survival time of patients after diagnosis is less than two years. This poor prognosis is attributable to the fact that GBM cells can rapidly invade the brain, a feature that is very difficult to attack with current treatment options. A better understanding of the molecular basis of GBM invasion, as well as how this phenomenon could be neutralized without damaging surrounding healthy cells, is therefore critically needed to develop more effective therapies. Accumulating evidence suggests that targeting the TrkB pathway may be a valid strategy to limit the growth, proliferation, and/or motility of aggressive cancer cells (Lawn et al., 2015).

The exploration of Trk inhibitors started a decade ago, but their number is limited and only a few have demonstrated antitumor efficacy in experimental preclinical models (Laetsch and Hong, 2021). TrkB inhibition using the small molecule inhibitor ANA-12, reduced medulloblastoma cell survival by inducing apoptosis (Thomaz et al., 2019). Other small-molecule pan-TRK inhibitors are currently under clinical development. Two of them have recently received FDA regulatory approval for the treatment of patients with solid tumors harboring an NTRK gene fusion; these are the selective TRK inhibitor larotrectinib and the multikinase inhibitor entrectinib (Laetsch and Hong, 2021). Nonetheless, there is always concern about acquired resistance to this first-generation of TRK inhibitors which may eventually lead to therapeutic failure.

U.S. Pat. No. 9,428,510 relates to azaindazole or diazaindazole type of compounds or a pharmaceutically acceptable salt or solvate of same, a stereoisomer or mixture of stereoisomers of same in any proportions, such as a mixture of enantiomers, as well as a pharmaceutical composition comprising such a compound, for use in the treatment of cancers associated with the overexpression of at least one Trk protein.

U.S. Pat. No. 10,377,818 describes methods of treating glioma. Aspects of the invention include administering a therapeutically-effective amount of an agent that inhibits the activity of one or more neuronal activity-regulated proteins selected from neuroligin-3, brain-derived neurotrophic factor (BDNF), or brevican, to a patient with glioma. In certain embodiments, the methods involve treating a neurological dysfunction, reducing the invasion of a glioma cell into brain tissue, and/or reducing the growth rate of glioma in the patient. It also provides methods for identifying an agent that modulates the mitotic index of a glial cell, and methods for stimulating the proliferation of a glial cell.

U.S. Patent Application Publication No. 2021/0023086 describes compounds and pharmaceutical compositions comprising the same compounds and the use of such compounds in the treatment of cancer. More particularly, it provides a method of treating cancer (e.g., Trk-associated cancer) by administration of one or more chemical Trk inhibitors and optionally an immunotherapy agent.

U.S. Patent Application Publication No. 2016/056822 and International Application Publication No. WO 2017/066434 relate to methods and treatments for improving cognitive functioning in patients in need. The methods comprise administering at least one BDNF-TrkB inhibitor. A61K31/55 Heterocyclic compounds having nitrogen as a ring hetero atom (e.g., guanethidine) or rifamycins having seven-membered rings (e.g., azelastine, pentylenetetrazole).

U.S. Patent Application Publication No. 2020/063890 and International Application Publication No. WO 2021/119056A1 relates to methods of treating cancer patients with RAS node or RTK targeted therapeutic agents. Described are methods for determining the functional status of G-protein coupled receptor (GPCR) signaling pathways in a diseased cell sample obtained from a subject to thereby select for therapeutic use in the subject a RAS node or receptor tyrosine kinase (RTK) targeted therapeutically. Also provided are methods for determining whether a GPCR signaling pathway is ultrasensitive in a diseased cell sample from a subject and methods of administering a selected RAS node or RTK targeted therapeutic agent.

U.S. Pat. No. 9,895,344 describes novel compounds and methods related to the activation of the TrkB receptor. The methods include administering in vivo or in vitro a therapeutically effective amount of 7,8-dihydroxyflavone (7,8-DHF) or derivative thereof. Specifically, methods and compounds for the treatment of disorders including neurologic disorders, neuropsychiatric disorders, and metabolic disorders. For example, for treating or reducing the risk of depression, anxiety, or obesity in a subject, and administering to the subject a therapeutically effective amount of 7,8-DHF or a derivative thereof. A further method of promoting neuroprotection in a subject also is described, which includes administering to the subject a therapeutically effective amount of 7,8-DHF or a derivative thereof.

U.S. Pat. No. 9,687,469 describes a pharmaceutical composition for the prevention and treatment of cancer with specific flavonoid-based compounds selected from among the groups of flavone, flavanone and flavanol, a method for the prevention and treatment of cancer and inflammation using the specific flavonoid-based pharmaceutical compositions, a method for isolating the flavonoid-based pharmaceutical compositions from raw plant material, and a method for synthesizing said specific flavonoid-based pharmaceutical compositions.

European Patent No. 2,044,935 relates to a composition comprising at least one non-psychotropic cannabinoid and/or at least one phenolic or flavonoid compound and/or Denbinobin and their uses for the prevention and treatment of gastrointestinal inflammatory diseases and for the prevention and treatment of gastrointestinal cancers. It also relates to a phytoextract obtained from the plant Cannabis sativa, more particularly from the selected variety CARMA.

There is a need for alternative therapies to overcome or mitigate at least some of the deficiencies of the prior art, and/or to provide a useful alternative.

### DESCRIPTION OF THE DRAWINGS

The present invention will be further understood from the following description with reference to the Figures, in which:

FIG. 1. Chemical structures of compounds described herein. (A) Chemical structures of cannflavins A, B, and C, as well as (B) ANA-12. (C) Table relating relevant flavonoids organized by: name, flavonoid class, chemical structure and impact of BDNF-induced Arc expression in mice primary cortical neurons as reported in Lalonde et al. (2017).

FIG. 2. Increasing concentrations of cannflavins A and B decrease Arc protein and mRNA levels. (A) Western blot and corresponding densitometry analysis showing Arc protein abundance when treated with various concentrations (0, 1, 5, 10, 20 μM) of cannflavin A (left) or cannflavin B (right). β-actin was used as loading control and graphs show the mean (±SEM) of Arc/β-actin ratio for each condition. Biological replicates: n=4-5. One-way ANOVA revealed a significant difference in the abundance of Arc with increasing cannflavin A and cannflavin B concentrations. Cannflavin A (F4,20=4.568, p=0.0088), Tukey's post-hoc test, *p<0.05, **p<0.001. Cannflavin B (F4,15=24.07, p<0.0001), Tukey's post-hoc test, *p<0.05, **p<0.001, ****p<0.0001. (B) Quantification of immunocytochemistry coverslips treated with various flavonoids (EGCG, daidzein and genistein). Quantification was completed by using a ratio of MAP2-positive cells with nuclear Arc above the 26 nuclear Arc pixel intensity in the control condition (BDNF treatment alone). Biological replicates: n=7. (C) Quantification of immunocytochemistry coverslips treated with various concentrations (0, 1, 5, 10, 20 μM) of cannflavin A (blue bars) or cannflavin B (red bars). Quantification was completed by using a ratio of MAP2-positive cells with nuclear Arc above the 26 nuclear Arc pixel intensity in the control condition (BDNF treatment alone). Biological replicates: n=3. (D) Quantitative real-time PCR Arc mRNA analysis using two Arc primer pairs shows a decrease in Arc mRNA transcripts when treated with 10 μM of cannflavin A or cannflavin B, relative to treated with BDNF alone.

FIG. 3. G-protein coupled receptors are not responsible for cannflavins signaling. Data for 320 GPCRs are presented as an average fold change from baseline upon compound addition. Application of quinpirole to D₂ receptor is used as a positive control in each plate. Compounds were used at 10 μM and all tests were run in quadruplicate.

FIG. 4. Cannflavins A and B decrease activation of downstream pathways of TrkB. (A) Simplified schematic of BDNF activation of TrkB receptors and downstream signaling pathways. B) Western blot analysis showing phosphorylation of TrkB when treated with BDNF and various concentrations (0, 1, 5, 10, 20 μM) of cannflavin A (left) or cannflavin B (right). β-actin was used a loading control. C) Western blot analysis showing phosphorylation of Mapk, Akt, and mTor proteins when treated with various concentrations (0, 1, 5, 10, 20 μM) of cannflavin A (left) or cannflavin B (right).

FIG. 5. Cannflavins A and B reduce BDNF-induced neurite outgrowths in immortalized neuroblastoma cells. (A) Neuroblastoma Neuro2a cells were transfected to stably express Ntrk2-Myc-FLAG. Immunocytochemistry was completed to validate that the cells were successfully transfected. Scale bar=20 μm. (B) Western blot analysis showing phosphorylation of TrkB and MapK in normal neuro2a's compared to Ntrk2-Myc-FLAG Neuro2as. β-actin was used as loading control. (C) Western blot analysis showing phosphorylation of TrkB and Mapk when treated with 10 μM of ANA-12, cannflavin A, or cannflavin B with the addition of BDNF (10 μg/mL). β-actin was used as loading control. (D) Phase-contrast images of Ntrk2-Myc-FLAG Neuro2as treated with or without BDNF (10 μg/mL) and 10 μM of ANA-12, cannflavin A, or cannflavin B. Scale bar=50 μm. 5 images per replicate, 3 replicates per treatment, and 2 biological replicates. Images were quantified by counting (E) viable cells, (F) total number of neurites, and (G) total number of cells bearing neurites twice the length of cell body. The addition of ANA-12 and cannflavins decreased the number of neurites and neurite-bearing cells. Data was analyzed by a one-way ANOVA (n=30; *p<0.05; **p<0.01; ***p<0.001; ***p<0.0001 vs. BDNF control condition). Graphs represent mean±SEM.

FIG. 6. Cannflavins A and B reduce the viability of Glioblastoma cells. Cell viability was measured using an AlamarBlue reaction after 24, 48, and 72 hours of treatment. Selective TrkB inhibition by (A) ANA-12, (B) cannflavin A, and (C) cannflavin B reduced cell viability of both A172 and U87 GBM cell lines in a dose- and time-dependent manner. Data was analyzed by a one-way ANOVA (ANA-12, n=15; cannflavins A and B, n=9; *p<0.05; **p<0.01; ***p<0.001; ***p<0.0001 vs. respective DMSO control condition). Graphs represent mean±SEM.

FIG. 7. TrkB inhibition by cannflavins A and B does not lead to cell necrosis in Glioblastoma cells. LDH cytotoxicity assay determined that treatment with ANA12, cannflavins A and B caused a slight increase in LDH release compared to DMSO after 24 hours in (A) A172 and (B) U87 GBM cells. (ANA-12, n=9; cannflavins A and B, n=6).

FIG. 8. Cannflavins A and B reduce the migration of Glioblastoma cells. Scratch analysis was performed on A172 and U87 GBM cells using a P1000 tip, then 10× images were taken at time zero and after 24 hours of treatment (A) ANA-12 at a minimal dose (10 μM); cannflavin images are not shown. (B) A172 GBM cells were grown into spheroids for 3 days prior to being plated on poly-L-lysine+laminin-coated plates and treated with minimal doses of ANA-12, cannflavin A, and cannflavin B for 24 hours.

FIG. 9. Cannflavins A and B reduce invasion of Glioblastoma cells. Boyden Chamber assay was performed on A172 GBM cells to analyze minimal doses (10 μM) of TrkB inhibitors on preventing GBM cell invasion. Cells were pretreated in low serum media for 24 hours. Bottoms of transwell inserts were coated with fibronectin and tops were coated with Matrigel prior to cells being seeded with treatment media (low serum; n=3 per treatment). After 24 hours, cells were cleaned off the top of the Matrigel, and cells that invaded into the pores of the transwell were stained with 1% Crystal Violet and (A) imaged at 20×. (B) Cells were counted per treatment and 10 μM ANA-12 and cannflavin B treatment caused a decrease in GBM cell invasion after 24 hours (n=15).

### SUMMARY

In accordance with an aspect, there is provided a method for treating and/or preventing cancer, the method comprising administering to a subject in need thereof a therapeutically effective and a pharmaceutically acceptable amount of a cannabis-derived flavonoid.

In an aspect, the cannabis-derived flavonoid inhibits Trk.

In an aspect, the cannabis-derived flavonoid is cannflavin A and/or cannflavin B and/or cannflavin C.

In an aspect, the cannabis-derived flavonoid decreases the activation of downstream pathways of TrkA, TrkB, and/or TrkC by disrupting signaling phosphorylation pathways of downstream kinases or proteins.

In an aspect, the cannabis-derived flavonoid reduces the viability of a cancerous cell in a dose and time-dependent manner.

In an aspect, the cannabis-derived flavonoid does not lead to cytotoxicity or cell necrosis.

In an aspect, the cannabis-derived flavonoid reduces cancerous cell migration.

In an aspect, the cannabis-derived flavonoid reduces cancerous cell invasion.

In an aspect, the cannabis-derived flavonoid limits activation of TrkB by the brain-derived neurotrophic factor (BDNF).

In an aspect, the cancer is a RTK/Trk-associated cancer.

In an aspect, the cancer comprises: brain cancers (e.g., glioblastoma multiforme, glioma, brain stem glioma), breast cancer, colorectal cancer, prostate cancer, pancreas cancer, ovarian cancer, lung cancer, bladder cancer, melanoma, thyroid cancer, head and neck cancers, uterine sarcoma, and/or neuroblastoma adrenocortical carcinoma.

In an aspect, the cancer comprises: bone cancer (e.g., osteosarcoma); central nervous system tumors (e.g. brain and spinal cord tumor; central nervous system embryonal tumors; ependymoma); bronchus cancer; cervical cancer; cutaneous T-cell lymphoma; endometrial cancer; esophageal cancer; eye cancer (e.g., retinoblastoma); fibrosarcoma; gallbladder cancer; heart cancer; hypopharyngeal cancer; islet cell tumor; kidney cancer; large cell neuroendocrine cancer; laryngeal cancer; leukemia (e.g., acute lymphoblastic leukemia; acute myeloid leukemia; chronic lymphocytic leukemia; chronic myelogenous leukemia); liver cancer; Burkitt lymphoma; Hodgkin's lymphoma; medulloblastoma; mesothelioma; mouth cancer; multiple myeloma; nephroma; pharyngeal cancer; salivary gland cancer; sarcoma (e.g., Ewing sarcoma, rhabdomyosarcoma, and undifferentiated sarcoma); small intestine cancer; stomach cancer; squamous cell carcinoma; squamous neck cancer; testicular cancer; urethral cancer; and vulvar cancer.

In an aspect, the method comprises administration of an effective dose of a pharmaceutical composition comprising the at least one cannabis-derived flavonoid, and optionally at least one pharmaceutically acceptable carrier.

In an aspect, the cannabis-derived flavonoid is administered separately, simultaneously, or sequentially with a Trk inhibitor, wherein the Trk inhibitor is optionally another cannabis-derived flavonoid, such as one or more of cannflavin A, cannflavin B, and cannflavin C.

In an aspect, the method further comprises administration of a flavonoid, such as: chrysoeriol, isocannflavin B, canaflone (FBL-03G), hesperetin, acacetin, apigenin, luteolin, chrysin, quercetin, kaempferol, 8-prenyl-kaempferol, galangin, 6-prenylnaringenin, hesperetin, vitexin, wogonin, and/or delphinidin.

In an aspect, the method further comprises administration of an anticancer agent.

In an aspect, the anticancer agent is a TrkA, TrkB, or TrkC inhibitor, for example: larotrectinib (LOXO-101), entrectinib (RXDX-101), selitrectinib (LOXO-195), repotrectinib (TPX-0005), cabozantinib (XL184), altiratinib (DCC-2701), sitravatinib (MGCD516), Taletrectinib (DS-6051b), merestinib, belizatinib (TSR-011), dovitinib (TKI-258), ONO-7579, crizotinib, ponatinib, nintedanib, GNF-4256, AZ64, cyclotraxin-B, or ANA-12.

In an aspect, the cannabis-derived flavonoid is substantially pure, for example, at least about 95%, about 96%, about 97%, about 98%, about 99%, about 99.5%, or about 99.9% pure.

In an aspect, the pharmaceutical composition is administered to the subject orally, intravenously, locally, or intrathecally.

In an aspect, the cannabis-derived flavonoid is formulated for sustained release.

In an aspect, the cannabis-derived flavonoid is obtained through organic chemical synthesis.

In an aspect, the cannabis-derived flavonoid is obtained through enzymatic synthesis.

In an aspect, the cannabis-derived flavonoid is obtained through in vivo biosynthesis by a recombinant method.

In an aspect, the cannabis-derived flavonoid is obtained through extraction and isolation from Cannabis sativa L., marijuana, or hemp.

In an aspect, the plant material from Cannabis sativa L., marijuana or hemp comprises a leaf, a root, a stem, a branch, a flower, an inflorescence, a fruit, a seed, a cell, a tissue culture, or a combination thereof.

In accordance with an aspect, there is provided a method for inhibiting Trk, the method comprising administering a cannabis-derived flavonoid.

In an aspect, the cannabis-derived flavonoid is cannflavin A and/or cannflavin B and/or cannflavin C.

In an aspect, the cannabis-derived flavonoid decreases the activation of downstream pathways of TrkA, TrkB, and/or TrkC by disrupting signaling phosphorylation pathways of downstream kinases or proteins.

In an aspect, the method is for treating and/or preventing a RTK/Trk-associated cancer.

In an aspect, the cancer comprises: brain cancers (e.g., glioblastoma multiforme, glioma, brain stem glioma), breast cancer, colorectal cancer, prostate cancer, pancreas cancer, ovarian cancer, lung cancer, bladder cancer, melanoma, thyroid cancer, head and neck cancers, uterine sarcoma, and/or neuroblastoma adrenocortical carcinoma.

In an aspect, the cancer comprises: bone cancer (e.g., osteosarcoma); central nervous system tumors (e.g. brain and spinal cord tumor; central nervous system embryonal tumors; ependymoma); bronchus cancer; cervical cancer; cutaneous T-cell lymphoma; endometrial cancer; esophageal cancer; eye cancer (e.g., retinoblastoma); fibrosarcoma; gallbladder cancer; heart cancer; hypopharyngeal cancer; islet cell tumor; kidney cancer; large cell neuroendocrine cancer; laryngeal cancer; leukemia (e.g., acute lymphoblastic leukemia, acute myeloid leukemia, chronic lymphocytic leukemia, chronic myelogenous leukemia); liver cancer; Burkitt lymphoma; Hodgkin's lymphoma; medulloblastoma; mesothelioma; mouth cancer; multiple myeloma; nephroma; pharyngeal cancer; salivary gland cancer; sarcoma (e.g., Ewing sarcoma, rhabdomyosarcoma, and undifferentiated sarcoma); small intestine cancer; stomach cancer; squamous cell carcinoma; squamous neck cancer; testicular cancer; urethral cancer; and vulvar cancer.

In an aspect, the cannabis-derived flavonoid reduces the viability of a cancerous cell in a dose and time-dependent manner.

In an aspect, the cannabis-derived flavonoid does not lead to cytotoxicity or cell necrosis.

In an aspect, the cannabis-derived flavonoid reduces cancerous cell migration.

In an aspect, the cannabis-derived flavonoid reduces cancerous cell invasion.

In an aspect, the cannabis-derived flavonoid limits activation of TrkB by BDNF.

In an aspect, the cannabis-derived flavonoid is substantially pure, for example, at least about 95%, about 96%, about 97%, about 98%, about 99%, about 99.5%, or about 99.9% pure.

In accordance with an aspect, there is provided a pharmaceutical or natural health product comprising cannflavin A and/or cannflavin B and/or cannflavin C for treating and/or preventing cancer.

In accordance with an aspect, there are provided cannflavins for preventing the normal increase in Arc protein by BDNF in a dose-dependent manner.

In an aspect, 10-20 μM of cannflavin A and 1-20 μM of cannflavin B results in significantly less Arc protein abundance than the level seen in a BDNF-alone control measure in vitro.

In accordance with an aspect, there are provided cannflavins for reducing Arc-positive neuronal abundance.

In accordance with an aspect, there are provided cannflavins for preventing BDNF from effectively stimulating its target receptor.

In accordance with an aspect, there are provided cannflavins for inhibiting TrkB receptors.

In accordance with an aspect, there are provided cannflavins for inhibiting BDNF-induced neurite outgrowth in TrkB overexpressed neuroblastoma cells.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from the detailed description.

### DETAILED DESCRIPTION

Described herein are methods for using cannabis-derived flavonoids in prepared formulation products, wherein the product prepared has uses in treating cancers. The compounds described in the present invention, corresponding to cannflavins A/B/C, in aspects have the property of inhibiting or modulating the activity of Trk proteins, in particular TrkB. Consequently, in aspects, said compounds can be used in the treatment of cancers associated with altered Trk signaling.

### Definitions

Unless defined otherwise, technical and scientific terms used herein have the same meaning as commonly understood by one of ordinary skill in the art to which this invention belongs. See for example Singleton et al., Dictionary of Microbiology and Molecular Biology 2^{nd} ed., J. Wiley & Sons (New York, N.Y. 1994); Sambrook et al., Molecular Cloning. A Laboratory Manual, Cold Springs Harbor Press (Cold Springs Harbor, NY 1989), each of which is incorporated herein by reference. For the purposes of the present invention, the following terms are defined below.

“Active” or “activity” for the purposes herein refers to a biological activity of a native or naturally-occurring molecule, wherein “biological” activity refers to a biological function (either inhibitory or stimulatory) caused by a native or naturally-occurring molecule.

Thus, “biologically active” or “biological activity” when used in conjunction with the flavonoids described herein refers to a molecule that exhibits or shares an effector function of the native flavonoid. For example, cannflavin A, cannflavin B, or cannflavin C described herein have the biological activity of treating cancers.

“Isolated” refers to a molecule that has been purified from its source or has been prepared by recombinant or synthetic methods and purified. Purified flavonoids are substantially free of contaminating components, such as THC, cannabinoids, and/or terpenes, for example.

“Substantially free” herein means less than about 5%, typically less than about 2%, more typically less than about 1%, even more typically less than about 0.5%, most typically less than about 0.1% contamination, such as with THC, cannabinoids, and/or terpenes.

As used herein, “treatment” or “therapy” is an approach for obtaining beneficial or desired clinical results. For the purposes described herein, beneficial or desired clinical results include, but are not limited to, alleviation of symptoms, diminishment of the extent of disease, stabilized (i.e., not worsening) state of disease, delay or slowing of disease progression, amelioration or palliation of the disease state, and remission (whether partial or total), whether detectable or undetectable. “Treatment” and “therapy” can also mean prolonging survival as compared to expected survival if not receiving treatment or therapy. Thus, “treatment” or “therapy” is an intervention performed with the intention of altering the pathology of a disorder. Specifically, the treatment or therapy may directly prevent, slow down or otherwise decrease the pathology of a disease or disorder such as inflammation, or may render the inflammation more susceptible to treatment or therapy by other therapeutic agents.

The terms “therapeutically effective amount”, “effective amount” or “sufficient amount” mean a quantity sufficient, when administered to a subject, including a mammal, for example, a human, to achieve the desired result, for example, an amount effective to treat inflammation. Effective amounts of the Cannflavin A, B, and C molecules described herein may vary according to factors such as the disease state, age, sex, and weight of the subject. Dosage or treatment regimens may be adjusted to provide the optimum therapeutic response, as is understood by a skilled person.

Likewise, an “effective amount” of the flavonoid compounds described herein refers to an amount sufficient to function as desired, such as to treat cancers.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use and that it is generally safe for administering to humans according to established governmental standards, including those promulgated by the United States Food and Drug Administration.

“Carriers” as used herein include pharmaceutically acceptable carriers, excipients, or stabilizers that are nontoxic to the cell or subject being exposed thereto at the dosages and concentrations employed. Often the pharmaceutically acceptable carrier is an aqueous pH buffered solution. Examples of pharmacologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, and dextrins; chelating agents such as EDTA; sugar alcohols such as mannitol and sorbitol; salt-forming counterions such as sodium; and/or non-ionic surfactants such as TWEEN™, polyethylene glycol (PEG), and PLURONICS™.

In understanding the scope of the present application, the articles “a”, “an”, “the”, and “said” are intended to mean that there are one or more of the elements. Additionally, the term “comprising” and its derivatives, as used herein, is intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having”, and their derivatives.

It will be understood that any embodiments described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known pharmaceutically acceptable additive, excipient, diluent, carrier, and the like. Typically, a composition consisting essentially of a set of components will comprise less than 5% by weight, typically less than 3% by weight, more typically less than 1% by weight of non-specified components.

It will be understood that any component defined herein as being included may be explicitly excluded from the claimed invention by way of proviso or negative limitation. For example, in embodiments, THC, cannabinoids, and/or terpenes are explicitly excluded from the compositions and methods described herein.

In addition, all ranges given herein include the end of the ranges and also any intermediate-range points, whether explicitly stated or not.

Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### Methods and Compositions

Described herein are various methods and related uses. For example, provided is a method for treating and/or preventing cancer. The method comprises administering to a subject in need thereof a therapeutically effective and a pharmaceutically acceptable amount of a cannabis-derived flavonoid.

In other aspects, provided is a method for inhibiting Trk, the method comprising administering a cannabis-derived flavonoid.

While any cannabis-derived flavonoid is contemplated, typically the cannabis-derived flavonoid inhibits Trk and, more typically, the cannabis-derived flavonoid is cannflavin A and/or cannflavin B and/or cannflavin C. It will be understood that any of these can be used alone or in combination with each other or with other cannabis components or with other non-cannabis components.

In aspects, the cannabis-derived flavonoid decreases the activation of downstream pathways of TrkA, TrkB, and/or TrkC by disrupting signaling phosphorylation pathways of downstream kinases or proteins.

In some aspects, the cannabis-derived flavonoid reduces the viability of a cancerous cell in a dose and time-dependent manner. In additional or alternative aspects, the cannabis-derived flavonoid does not lead to cytotoxicity or cell necrosis. In additional or alternative aspects, the cannabis-derived flavonoid reduces cancerous cell migration and/or cancerous cell invasion.

In aspects, the cannabis-derived flavonoid limits activation of TrkB by the BDNF.

While the treatment or prevention of any cancer is contemplated herein, typically the cancer is a RTK/Trk-associated cancer. For example, the cancer typically comprises brain cancers (e.g., glioblastoma multiforme, glioma, brain stem glioma), breast cancer, colorectal cancer, prostate cancer, pancreas cancer, ovarian cancer, lung cancer, bladder cancer, melanoma, thyroid cancer, head and neck cancers, uterine sarcoma, and/or neuroblastoma adrenocortical carcinoma.

In aspects, the cancer comprises bone cancer (e.g., osteosarcoma); central nervous system tumors (e.g. brain and spinal cord tumor; central nervous system embryonal tumors; ependymoma); bronchus cancer; cervical cancer; cutaneous T-cell lymphoma; endometrial cancer; esophageal cancer; eye cancer (e.g., retinoblastoma); fibrosarcoma; gallbladder cancer; heart cancer; hypopharyngeal cancer; islet cell tumor; kidney cancer; large cell neuroendocrine cancer; laryngeal cancer; leukemia (e.g., acute lymphoblastic leukemia; acute myeloid leukemia; chronic lymphocytic leukemia; chronic myelogenous leukemia); liver cancer; Burkitt lymphoma; Hodgkin's lymphoma; medulloblastoma; mesothelioma; mouth cancer; multiple myeloma; nephroma; pharyngeal cancer; salivary gland cancer; sarcoma (e.g., Ewing sarcoma; rhabdomyosarcoma; and undifferentiated sarcoma); small intestine cancer; stomach cancer; squamous cell carcinoma; squamous neck cancer; testicular cancer; urethral cancer; and vulvar cancer.

Thus in aspects, the methods described herein comprise administration of an effective dose of a pharmaceutical composition comprising the at least one cannabis-derived flavonoid, and optionally at least one pharmaceutically acceptable carrier. It will be understood that the cannabis-derived flavonoid may be administered separately, simultaneously, or sequentially with a Trk inhibitor. The Trk inhibitor may optionally be another cannabis-derived flavonoid, such as one or more of cannflavin A, cannflavin B, and cannflavin C or it may be any Trk inhibitor known to the skilled person.

In aspects, the method further comprises administration of a flavonoid, such as: chrysoeriol, isocannflavin B, canaflone (FBL-03G), hesperetin, acacetin, apigenin, luteolin, chrysin, quercetin, kaempferol, 8-prenyl-kaempferol, galangin, 6-prenylnaringenin, hesperetin, vitexin, wogonin, and/or delphinidin. In additional or alternative aspects, the method further comprises administration of an anticancer agent, such as a TrkA, TrkB, or TrkC inhibitor, for example: larotrectinib (LOXO-101), entrectinib (RXDX-101), selitrectinib (LOXO-195), repotrectinib (TPX-0005), cabozantinib (XL184), altiratinib (DCC-2701), sitravatinib (MGCD516), Taletrectinib (DS-6051b), merestinib, belizatinib (TSR-011), dovitinib (TKI-258), ONO-7579, crizotinib, ponatinib, nintedanib, GNF-4256, AZ64, cyclotraxin-B, or ANA-12.

In aspects, the cannabis-derived flavonoid is substantially pure, for example, at least about 95%, about 96%, about 97%, about 98%, about 99%, about 99.5%, or about 99.9% pure and is administered to the subject by any known method, such as, for example, orally, intravenously, locally, or intrathecally.

The cannabis-derived flavonoid may be formulated in any known way. For example, in some aspects, the cannabis-derived flavonoid is formulated for sustained release.

The cannabis-derived flavonoid may be made/isolated by any known method. In some aspects, the cannabis-derived flavonoid is obtained through organic chemical synthesis. In some aspects, the cannabis-derived flavonoid is obtained through enzymatic synthesis. In some aspects, the cannabis-derived flavonoid is obtained through in vivo biosynthesis by a recombinant method. In some aspects, the cannabis-derived flavonoid is obtained through extraction and isolation from Cannabis sativa L., marijuana, or hemp. The plant material from Cannabis sativa L., marijuana or hemp from which the cannabis-derived flavonoid may be obtained in aspects comprises a leaf, a root, a stem, a branch, a flower, an inflorescence, a fruit, a seed, a cell, a tissue culture, or a combination thereof.

Also described herein is a pharmaceutical or natural health product comprising cannflavin A and/or cannflavin B and/or cannflavin C for treating and/or preventing cancer.

The cannflavins described herein may be used in many different ways. For example, the cannflavins are, in aspects, used for for preventing the normal increase in Arc protein by BDNF in a dose-dependent manner. For example, from about 10 to about 20 μM of cannflavin A and from about 1 to about 20 μM of cannflavin B may result in significantly less Arc protein abundance than the level seen in a BDNF-alone control measure in vitro.

In additional or alternative aspects, the cannflavins described herein may be used for reducing Arc-positive neuronal abundance, for preventing BDNF from effectively stimulating its target receptor, for inhibiting TrkB receptors, and/or for inhibiting BDNF-induced neurite outgrowth in TrkB overexpressed neuroblastoma cells.

Also described herein are pharmaceutical compositions comprising at least one cannabis-derived flavonoid, cannflavin A and/or cannflavin B and/or cannflavin C.

Typically, methods to produce or obtain cannflavin A and/or cannflavin B and/or cannflavin C comprise organic chemical synthesis, in vitro enzymatic synthesis, in vivo biosynthesis by a recombinant method, or by extraction and isolation from cannabis tissues.

Typically, a recombinant method involves endogenously expressing and/or engineering to express at least one polypeptide in a host cell or organism, such as a bacterium, an archaeon, a yeast, a protozoon, an alga, a fungus, or a plant, including single cells and cell cultures of any thereof for enzymatically acting on a molecule present in the host cell or organism or its cell culture medium.

Typically, isolation of flavonoids from cannabis involves extracting the plant material with a polar solvent, pure or in an aqueous solution, and separating the flavonoids by affinity and/or molecular mass in one or more steps of column (chromatography) or batch systems. The plant material can be any cannabis species, including marijuana and hemp. For example, Cannabis sativa, Cannabis indica, Cannabis ruderalis, and individual strain or combinations of strains within any of these species or combination of species thereof may serve as the source of cannflavins. Typically, cannflavins are derived from Cannabis sativa L. Similarly, it will be understood that the cannflavins may be derived from any plant source, including a leaf, a root, a stem, a branch, a flower, an inflorescence, a fruit, a seed, a cell, a tissue culture, or a combination thereof.

The compositions may be formulated for use by a subject, such as a mammal, including a human. Compositions comprising cannflavin A and/or cannflavin B and/or cannflavin C described herein may comprise about 0.00001% to about 99% by weight of the active and any range there-in-between, such as from about 0.00001%, about 0.0001%, about 0.001%, about 0.01%, about 0.1%, about 0.5%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.7%, or about 99.9%, to about 0.0001%, about 0.001%, about 0.01%, about 0.1%, about 0.5%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.7%, about 99.9%, about 99.99%. For example, typical doses may comprise from about 0.1 μg to about 100 μg of the molecules described herein per 300 mg dose, such as about 0.5 μg, about 1 μg, about 2 μg, about 3 μg, about 4 μg, about 5 μg, about 6 μg, about 7 μg, about 8 μg, about 9 μg, about 10 μg, about 25 μg, about 50 μg, or about 75 μg per 300 mg dose, such as from about 0.1 μg to about 10 μg, or from about 1 μg to about 5 μg, or from about 1 μg to about 2 μg per 300 mg dose (and all related increments and percentages by weight).

The compositions described herein may be used in any suitable amount, but are typically provided in doses comprising from about 1 to about 10000 ng/kg, such as from about 1 to about 1000, about 1 to about 500, about 10 to about 250, or about 50 to about 100 ng/kg, such as about 1, about 10, about 25, about 50, about 75, about 100, about 150, about 200, about 250, about 300, or about 500 ng/kg. In other aspects, the compositions described herein are provided in doses of from about 1 to about 10000 mg per dose, such as from about 1 to about 1000, about 1 to about 500, about 10 to about 250, or about 50 to about 100 mg, such as about 1, about 10, about 25, about 50, about 75, about 100, about 150, about 200, about 250, about 300, about 400, about 500 mg, about 600 mg, about 700 mg, about 800 mg, about 900 mg, or about 1000 mg. For example, in some aspects, cannflavin A is provided at about 500 mg per dose, cannflavin B is provided at about 400 mg per dose, and cannflavin C is provided at about 100 mg per dose. In aspects, cannflavin A, cannflavin B, and cannflavin C are provided together in a ratio, such as a ratio of about 5:4:1.

In other aspects, the compositions described herein are dosed so as to obtain about a 0.01 μM to about a 100 μM target concentration in blood of a human, such as from about 0.01 μM, about 0.05 μM, about 0.1 μM, about 0.2 μM, about 0.3 μM, about 0.4 μM, about 0.5 μM, about 0.6 μM, about 0.7 μM, about 0.8 μM, about 0.9 μM, about 1 μM, about 2 μM, about 3 μM, about 4 μM, about 5 μM, about 6 μM, about 7 μM, about 8 μM, about 9 μM, about 10 μM, about 20 μM, about 30 μM, about 40 μM, about 50 μM, about 60 μM, about 70 μM, about 80 μM, or about 90 μM to about 0.05 μM, about 0.1 μM, about 0.2 μM, about 0.3 μM, about 0.4 μM, about 0.5 μM, about 0.6 μM, about 0.7 μM, about 0.8 μM, about 0.9 μM, about 1 μM, about 2 μM, about 3 μM, about 4 μM, about 5 μM, about 6 μM, about 7 μM, about 8 μM, about 9 μM, about 10 μM, about 20 μM, about 30 μM, about 40 μM, about 50 μM, about 60 μM, about 70 μM, about 80 μM, about 90 μM, or about 100 μM. For example, typically cannflavins A, B and C are dosed so as to obtain about a 2 μM target concentration. The compositions may be administered over a period of hours, days, weeks, or months, depending on several factors, including the severity and type of the tumor or other condition being treated, whether a recurrence is considered likely, etc. The administration may be constant, e.g., constant infusion over a period of hours, days, weeks, months, etc. Alternatively, the administration may be intermittent, e.g., the composition may be administered once a day over a period of days, once an hour over a period of hours, or any other such schedule as deemed suitable.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in “Handbook of Pharmaceutical Additives” (compiled by Michael and Irene Ash, Gower Publishing Limited, Aldershot, England (1995)). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids. In this regard, reference can be made to U.S. Pat. No. 5,843,456 (the entirety of which is incorporated herein by reference).

Pharmaceutically acceptable carriers are well known to those skilled in the art and include, for example, sterile saline, lactose, sucrose, calcium phosphate, gelatin, dextrin, agar, pectin, peanut oil, olive oil, sesame oil, cannabis oil, and water. Furthermore, the composition may comprise one or more stabilizers such as, for example, carbohydrates including sorbitol, mannitol, starch, sucrose, dextrin, and glucose, proteins such as albumin or casein, and buffers like alkaline phosphates.

The compositions described herein can, in embodiments, be administered for example, by parenteral, intravenous, subcutaneous, intradermal, intramuscular, intracranial, intrathecal, intraorbital, ophthalmic, intraventricular, intracapsular, intraspinal, intracisternal, intraperitoneal, intranasal, intrarectal, intravaginal, aerosol, oral, topical, or transdermal administration. Typically, the compositions of the invention are administered orally or intravenously.

It is understood by one of skill in the art that the compositions described herein can be used in conjunction with known therapies for prevention and/or treatment of cancers in subjects and/or with compositions for preventing cancer progression or other compositions. The compositions described herein may, in embodiments, be administered in combination, concurrently or sequentially, with conventional treatments for cancers, including chemotherapy or radiotherapy procedures, for example. The compositions described herein may be formulated together with such conventional treatments when appropriate.

The compositions comprising the flavonoids described herein are expected to exhibit anticancer activity. Other flavonoid molecules have been reported as cancer chemopreventive agents. For example, consumption of the flavonol quercetin is inversely associated with the incidence of prostate, lung, stomach, and breast cancers. Ingestion of resveratrol also seems to lower the risk of developing lung, endometrium, esophagus, stomach, and colon cancers. Different cellular inhibitory activities are amongst the proposed mechanisms by which flavonoids have an effect on the initiation and promotion stages of carcinogenicity.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1

### Introduction

It has now been found that cannflavins—a class of flavonoid molecules that accumulates in the Cannabis sativa plant—can prevent TrkB activation. We have focused on identifying specific mechanisms of action of these two related cannabis-derived metabolites in neuronal cells and completing a systematic preclinical characterization of cannflavins A and B to position these small molecules as valid therapeutic agents against cancer cells, such as glioblastoma (GBM) cells. In vitro experiments with GBM cell lines including cell viability assay, scratch migration assay, and trans-well invasion assay were used to determine the therapeutic effects ANA-12 and cannflavins have against key cancer, such as GBM, hallmarks.

Previously, we published a chemogenomic analysis that aimed at identifying small molecule modulators of Activity-regulated cytoskeleton-associated protein (Arc), which is a key regulator of neuroplasticity and cognitive functions (Bramham et al., 2010; Korb et al., 2011; Kedrov et al., 2019). Our approach in that project exploited the ability of the growth factor BDNF to promote abundant Arc mRNA expression followed by nuclear accumulation of the protein product in mouse primary cortical neurons via activation of TrkB receptor (Lalonde et al., 2017). Here, we have adapted this assay to test the two cannflavins and found evidence of TrkB signaling interference by both molecules. These results then led us to complete a secondary high-throughput screen to test the possible agonist activity of these flavonoids on G-coupled-protein receptors (GPCRs), as well as standard biochemical analyses to confirm the influence of cannflavins and pinpoint their target engagement. Without wishing to be bound by theory, these specific efforts support a model where cannflavins interfere with TrkB activity through direct inhibitory binding with the receptor. Finally, an image-based cellular test with immortalized Neuro2a cells ectopically expressing TrkB allowed us to demonstrate the capacity of cannflavins to block BDNF-induced neurite outgrowth. In sum, our study supports the classification of cannflavins as inhibitors of TrkB receptor signaling.

### Methods

### Cell Culture and Transfection.

Developing cerebral cortex from E16.5 CD-1 mouse embryos were dissected and then dissociated in trypsin solution for 15 min followed by three washes with phosphate-buffered saline (PBS). Trypsinized tissue was gently triturated to produce single cell suspension. Next, cells were seeded in poly-L-lysine/laminin coated 6-well plates at a density of 1.5×10⁶ per well and maintained in Neurobasal medium containing B27 supplement (2%, Invitrogen, Grand Island, NY), penicillin (50 U/ml, Invitrogen), streptomycin (50 μg/ml, Invitrogen) and glutamine (1 mM, Sigma). For experiments involving BDNF (PeproTech, Rocky Hill, NJ), the growth factor was added directly to the culture medium at a final concentration of 100 ng/ml for the indicated period of time. Preparation of mouse primary cortical neuron cultures was approved by the University of Guelph Animal Care Committee and carried out according to institutional guidelines.

For neurite outgrowth assay, Neuro2a cells were cultured in DMEM [supplemented with 10% HyClone FetalClone II serum (Cytiva, Global Life Sciences Solutions, Marlborough, MA), penicillin (50 units/ml), and streptomycin (50 μg/ml)] and transfected overnight using Lipofectamine 2000 (Invitrogen) according to the manufacturer's protocol.

### Antibodies, Plasmid, and Pharmacological Compounds.

The anti-Arc rabbit polyclonal affinity purified antibody (#156 003) was purchased from Synaptic Systems (Goettingen, Germany). The antibodies recognizing p42 Mapk (Erk2, sc-1647), was from Santa Cruz Biotechnology (Santa Cruz, CA). The antibodies recognizing phosphorylated TrkA^{Tyr490}/TrkB^{Tyr516} (#4619), phosphorylated p44/42 Mapk (Erk1/2^{Thr202/Tyr204}, #4370), AKT (#4691), phosphorylated AKT^{Thr308} (#2965), phosphorylated AKT^{Ser473} (#4060), mTor (#2983), phosphorylated mTor^{Ser2448} (#2971), and phosphorylated rpS6^{Ser240/244} (#2215) were acquired from Cell Signaling Technology (Beverly, MA). The antibodies recognizing TrkB (MAB397) were acquired from R&D Systems (Minneapolis, MN). The antibodies recognizing b-actin (A1978) and M2 FLAG (F1804) antibodies were from Sigma-Aldrich (St-Louis, MO), while the Map2 (AB5543) antibody was purchased from EMD Millipore Corps (Billerica, MA). Finally, cross-absorbed horseradish peroxidase-conjugated secondary antibodies were from Invitrogen.

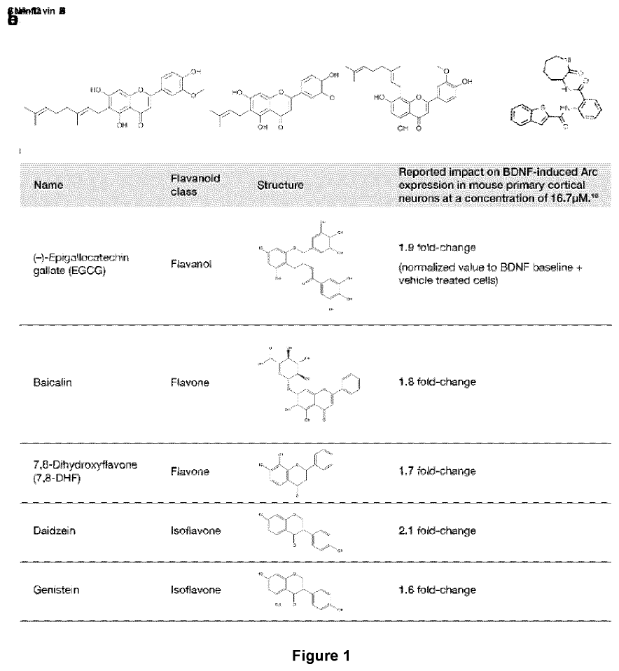

The pCMV6-Ntrk2-Myc-DDK (FLAG) plasmid (MR226130) was purchased from OriGene Biotechnologies (Rockville, MD). ECGC, genistein, and daidzein were from Sigma-Aldrich. ANA-12 (FIG. 1b) was from Tocris Bioscience (Bristol, UK) and U0126 was from Biosciences (Thermo Fisher).

The synthesis and purification of cannflavins A and B were produced using the method of Rea and colleagues (2019). Briefly, Cannabis sativa L. prenyltransferase 3 (CsPT3) was recombinantly expressed in Saccharomyces cerevisiae and the microsomal fraction containing CsPT3 was collected for in vitro enzyme assays. Assays containing 200 μM chrysoeriol, 400 μM GPP or DMAPP, 1 mg/mL of microsomal CsPT3, and 10 mM MgCl₂ in 100 mM Tris-HCl buffer were conducted at 37° C. for 120 min and terminated with the addition of 20% formic acid. Cannflavin products were extracted with three volumes of ethyl acetate, the organic layer was dried under N₂ gas and resuspended in methanol. The products were purified by HPLC on an Agilent 1260 Infinity system with a Waters SPHERISORB 5 μm ODS2 column, eluted with a 20 min linear gradient from 45% to 95% methanol in water containing 0.1% formic acid. Product identities were confirmed via LC-MS according to published methods (Rea et al., 2019) (Supplementary FIG. 1). Cannflavin products from multiple in vitro reactions were pooled and dried under nitrogen gas and resuspended in dimethyl sulfoxide (DMSO). Concentration of the final product was confirmed via HPLC eluted with a five-minute linear gradient from 80% to 95% methanol followed by a five-minute linear gradient from 95% to 100% methanol. Cannflavins were quantified by absorption at 340 nm relative to authentic standards.

### Western Blotting.

For western blot analyses, cells were collected by scraping in ice-cold radioimmunoprecipitation assay (RIPA) buffer (50 mM tris-HCl [pH 8.0], 300 mM NaCl, 0.5% Igepal-630, 0.5% deoxycholic acid, 0.1% SDS, 1 mM EDTA) supplemented with a cocktail of protease inhibitors (Complete Protease Inhibitor without EDTA, Roche Applied Science, Indianapolis, IN) and phosphatase inhibitors (Phosphatase Inhibitor Cocktail 3, Sigma-Aldrich). One volume of 2× Laemmli buffer (100 mM tris-HCl [pH 6.8], 4% SDS, 0.15% bromophenol blue, 20% glycerol, 200 mM b-mercaptoethanol) was added and the extracts were boiled for 5 min. Samples were adjusted to an equal concentration after protein concentrations were determined using the BCA assay (Pierce, Thermo Fisher Scientific). Lysates were separated using SDS-PAGE (polyacrylamide gel electrophoresis) and transferred to a nitrocellulose membrane. After transfer, the membrane was blocked in TBST (tris-buffered saline and 0.1% Tween 20) supplemented with 5% nonfat powdered milk and probed with the indicated primary antibody at 4° C. overnight. After washing with TBST, the membrane was incubated with the appropriate secondary antibody and visualized using enhanced chemiluminescence (ECL) reagents according to the manufacturer's guidelines (Pierce, Thermo Fisher Scientific).

The following procedure was used to quantify western blot analyses. First, equal quantity of protein lysate was analyzed by SDS-PAGE for each biological replicate. Second, the exposure time of the film to the ECL chemiluminescence was the same for each biological replicate. Third, all the exposed films were scanned on a HP Laser Jet Pro M377dw scanner in grayscale at a resolution of 300 dpi. Fourth, the look-up table (LUT) of the scanned tiff images was inverted and the intensity of each band was individually estimated using the selection tool and the histogram function in Adobe Photoshop CC 2021 software. Finally, the intensity of each band was divided by the intensity of its respective loading control (b-actin) to provide the normalized value used for statistical analysis.

### Immunocytochemistry.

Indirect immunofluorescence detection of antigens was carried out using cortical neurons cultured on poly-L-lysine/laminin coated coverslips in 24-well plates at a density of 0.1′10⁶ per well. After experimental treatment, cells were washed twice with phosphate-buffered saline (PBS) and fixed for 30 min at room temperature with 4% paraformaldehyde in PBS. After fixation, cells were washed twice with PBS, permeabilized with PBST (PBS and 0.25% Triton X-100) for 20 min, blocked in blocking solution (5% goat non-immune serum in PBS) for another 30 min, and finally incubated overnight at 4° C. with the first primary antibody in blocking solution. The next day, coverslips were extensively washed with PBS and incubated for 2 hours at room temperature in the appropriate fluorophore-conjugated secondary antibody solution [Alexa Fluor 488-, Alexa Fluor 594, or Alexa Fluor 647-conjugated secondary antibody (Molecular Probes, Invitrogen) in blocking solution]. After washes with PBS, the coverslips were incubated again overnight in primary antibody solution for the second antigen, and the procedure for conjugation of the fluorophore-conjugated secondary antibody was repeated as above. Finally, cell nuclei were counterstained with 4′,6-diamidino-2-phenylindole (DAPI), and coverslips were mounted on glass slides with ProLong Antifade reagent (Invitrogen).

Cells cultured on coverslips from three independent biological replicates were imaged with a Nikon Eclipse Ti2-E inverted microscope equipped with a motorized stage, image stitching capability, and a 60× objective (Nikon Instruments, Melville, NY). Image analysis was performed with ImageJ and NIS Elements and the following procedure was used to quantify nuclear Arc level in response to BDNF-TrkB signaling. First, original raw tiff files were opened and the nucleus of all neurons in the image was located based on MAP2 immunostaining, then average pixel intensity corresponding to Arc immunofluorescence was measured for a 30-pixel spot positioned at the center of the nuclear compartment. Second, for each measure of Arc nuclear immunofluorescence pixel intensity, a measure of background pixel intensity from the same image channel was acquired and subsequently subtracted from the Arc nuclear immunofluorescence pixel intensity value. Finally, Arc immunofluorescence signal from untreated samples was used to establish an objective threshold (two standard deviations above the nuclear Arc immunofluorescence signal averaged from a representative population of untreated neurons) and allow comparison of nuclear Arc expression between different experimental conditions.

### Real-Time Reverse Transcriptase PCR.

After experimental treatment, total RNA was isolated from primary cortical neuron cultures using the TRIzol method (Invitrogen). The concentration of total RNA was measured using a NanoDrop ND-8000 spectrophotometer (Thermo Fisher Scientific) and first-strand complementary DNA (cDNA) was synthesized using the iScript cDNA Synthesis Kit (Bio-Rad, Hercules, CA). Real-time PCRs were performed using gene-specific primers and monitored by quantification of SYBR Green I fluorescence using a Bio-Rad CFX96 Real-Time Detection System. Expression was normalized against Gapdh expression. The relative quantification from three biological replicates was calculated using the comparative cycle threshold (ΔΔC_{T}) method.

Primers for real-time reverse transcription PCR experiments were: Arc primer pair one, 5′-TAGCCAGTGACAGGACCCAG-3′ (SEQ ID NO: 1) (forward) and 5′-CAGCTCAAGTCCTAGTTGGCAAA-3′ (SEQ ID NO: 2) (reverse); Arc primer pair two, 5′-CGCCAAACCCAATGTGATCCT-3′ (SEQ ID NO: 3) (forward) and 5′-TTGGACACTTCGGTCAACAGA-3′ (SEQ ID NO: 4) (reverse); Gapdh, 5′-ATGACCACAGTCCATGCCATC-3′ (SEQ ID NO: 5) (forward) and 5′-CCAGTGGATGCAGGGATGATGTTC-3′ (SEQ ID NO: 6) (reverse).

### PRESTO-Tango GPCR Assay.

Parallel receptorome expression and screening via transcriptional output, with transcription activation following arresting translocation (PRESTO-Tango) was used to assess cannflavin A and cannflavin B potential to stimulate G protein-coupled receptors (GPCRs) according to published method (Kroeze et al., 2015). Overall, 320 distinct nonolfactory human GPCRs were tested.

### Neurite Outgrowth Assessment.

Neuro2A cells transfected with a pCMV6-Ntrk2-Myc-DDK (FLAG) construct were selected with G418 (Geneticin) to produce a stable cell line that constitutively expresses tagged-TrkB. For neurite outgrowth assessment, cells were seeded on 15 mm glass coverslips in 12-well plates at a density of 2.0×10⁴ per well. Cells were treated with BDNF (1 nM) in presence of cannflavins (10 μM), ANA-12 (10 μM), or vehicle control (DMSO). Phase contrast digital images were collected with a 20× objective 48 h after plating and the total number of viable cells, total number of neurites, and cells with neurites longer than 2 cells in diameter were counted (five fields per dish, three wells per condition).

### Statistics.

Unless mentioned otherwise, all results represent the mean±SEM from at least three independent experiments. ANOVA followed by Tukey's post hoc test for multiple comparisons were performed where indicated.

### Results

### Impact of Cannflavins on BDNF-Induced Arc Expression in Mouse Primary Cortical Neurons.

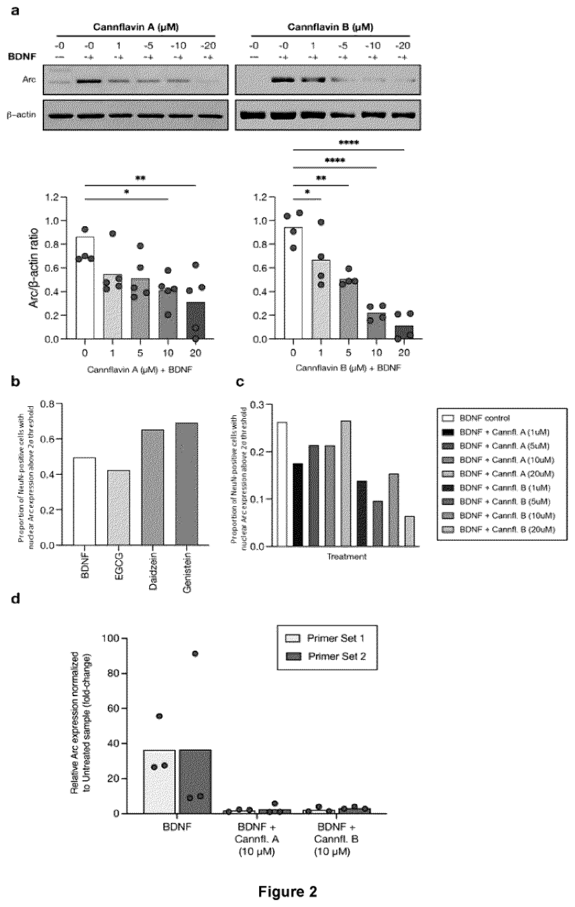

A decade ago, researchers identified a TrkB ligand ANA-12 (FIG. 1B) that selectively and directly binds and prevents the activation of the receptor and its downstream processes (Cazorla et al., 2011). Previously, we completed a chemogenomic screen that allowed us to identify a diverse set of Arc expression modifiers effective in differentiated mouse cortical neurons (Lalonde et al., 2017). As part of this collection of molecules, five distinct flavonoids (FIG. 1C)—namely (−)-epigallocatechin (ECGC), baicalin (BAI), 7,8-dihydroxyflavone (7,8-DHF), daidzein, and genistein-were found to enhance nuclear Arc protein level above the control measure when co-applied at a final concentration of 16.7 μM with recombinant BDNF for 6 h. Searching for a possible explanation to this phenomenon, we noticed different studies that had linked each of these five flavonoids to either enhancement of BDNF and/or TrkB mRNA expression, or to the potentiation of downstream TrkB-dependent signaling (Pan et al., 2012; Gundimeda et al., 2014; Ding et al., 2018; Lu et al., 2019). Based on this information, we then hypothesized that cannflavin A and cannflavin B could act in a similar fashion and promote Arc protein abundance when added to cultured cortical neurons stimulated with exogenous BDNF. Unexpectedly, though, western blot analysis assessing BDNF-induced Arc expression in conjunction with cannflavins for concentrations ranging between 1 to 20 μM revealed an opposite result. Specifically, we found that application of cannflavins to cell culture media prevented the normal increase in Arc protein by BDNF in a dose-dependent manner where 10-20 μM of cannflavin A and all tested concentrations (1-20 μM) of cannflavin B resulted in significantly less Arc protein abundance than the level seen in the BDNF-alone control measure (FIG. 2A). To confirm this effect, we repeated the experiment using fluorescent immunocytochemistry and quantified nuclear Arc changes, as we had done previously in our chemogenomic screen (Lalonde et al., 2017). Here, we also included the flavonol ECGC, and others with the isoflavone daidzein and genistein to replicate our earlier screening results. As predicted, cells that were co-treated with BDNF and 10 μM of ECGC, daidzein, or genistein presented a moderate increase in the percentage of nuclei with Arc expression above threshold in comparison to the BDNF-alone control (FIG. 2B). However, consistent with the initial western blotting data, cultures treated with BDNF and 10 μM cannflavins presented similar overall trends in the reduction of Arc-positive neuronal abundance in comparison to the unstimulated control (FIG. 2C). Together, these results confirm the discrepancy existing between cannflavins and the other flavonoids found in our earlier screen focusing on BDNF-induced Arc expression modifiers.

Next, to know whether the cannflavins' influence on TrkB signaling and Arc protein levels was produced before or after transcriptional events, we performed a quantitative polymerase chain reaction (qPCR) experiment. Here, comparison of Arc mRNA abundance between untreated, BDNF-alone control, and BDNF with cannflavin A (10 μM) or cannflavin B (10 μM) samples clearly indicated that cannflavins prevent induction of Arc mRNA expression (FIG. 2D), therefore suggesting that the effect of these compounds must occur somewhere between the activation of TrkB receptors by BDNF and the activation of transcriptional machinery involved in Arc expression.

### Evaluating Agonist Potential of Cannflavins on Tango GPCR Assay.

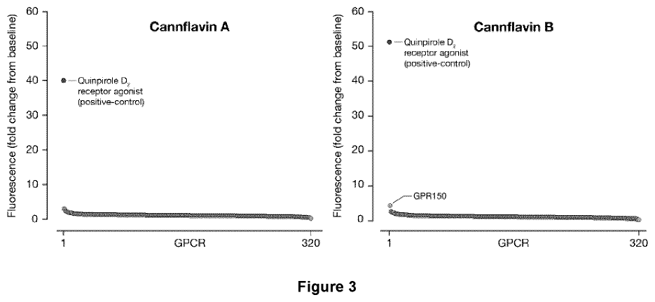

Considering on one hand examples for transactivation crosstalk between GPCRs and receptor tyrosine kinases, including TrkB (Rajagopal et al., 2004; 2006; El Zein et al., 2007), and recent evidence for GPCR modulation/self-association by flavonoids on the other (Herrera-Hernandez et al., 2017; Ortega et al., 2019), we speculated that perhaps one mechanism by which cannflavins could interfere with Arc expression in cortical neurons involves activation of a G protein signal that trans-inactivates the function of molecular cascades downstream of TrkB receptors responsible for Arc expression. To find support for this scenario, we then interrogated the influence of cannflavins on the GPCRome en masse using the PRESTO-Tango assay—an unbiased high-throughput screening approach adapted to identify agonist activity of agents towards the large family of GPCRs (Kroeze et al, 2015). Interestingly, probing for cannflavin A revealed no effect on any of the 322 different GPCRs tested while cannflavin B was found to produce only a weak increase (4.4 fold-change) of GPR150 activity from baseline, a small effect in comparison to the one seen with the positive control (51.3 fold-change, dopamine D2 receptor stimulated by quinpirole) (FIG. 3). Faced with these results, we then decided to abandon the possibility of G protein trans-inactivation of TrkB and focused instead our attention on the possibility that cannflavins act more directly on the TrkB receptor and/or its downstream signaling.

### Elucidating Cannflavins Effects on TrkB Signaling.

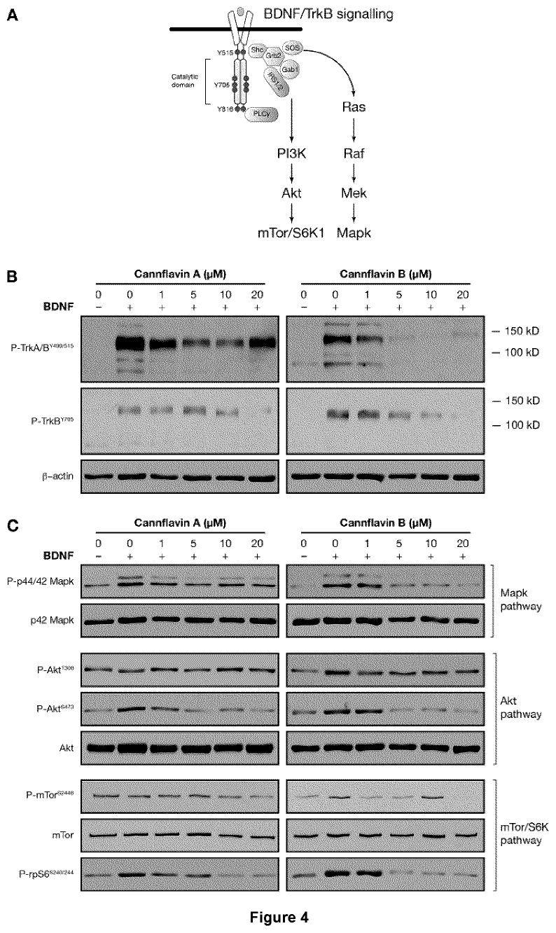

BDNF binding to extracellular domains of TrkB stimulates receptor dimerization and phosphorylation of intracellular tyrosine residues followed by the recruitment of pleckstrin homology (PH) and SH2 domain-containing proteins such as FRS2, Shc, SH2B, and SH2B2 which regulate distinct concurrent signaling cascades (Meakin et al., 1999; Qian et al., 1998). In order to establish whether cannflavins interfere with the activation of TrkB receptors by BDNF in primary cortical neurons, we used a western blotting approach and probed lysates with a P-TrkA/P-TrkB antibody. This approach revealed that, indeed, cannflavins can prevent BDNF from effectively stimulating its target receptor (FIG. 4B). To further support this result, we tested the activation of signaling pathways that are likely regulated downstream of TrkB, including the Ras-Raf-MEK-MAPK and the PI3K/AKT/mTOR cascades (Huang et al., 2003; Kowianski et al., 2018) (FIG. 4A). Interestingly, our analyses revealed that both cannflavin A and cannflavin B sharply reduced the normal increase in P-MAPK, and P-Akt, P-mTOR, and P-rpS6 levels (FIG. 4C). The fact that there were alterations in these two molecular pathways, which to a great extent occur in parallel with limited cross-interact, strongly suggest that the cannflavins must have an effect at an early stage in TrkB signal activation.

### Functional Characterization TrkB Inhibition by Cannflavins on BDNF-Dependent Neurite Outgrowth.

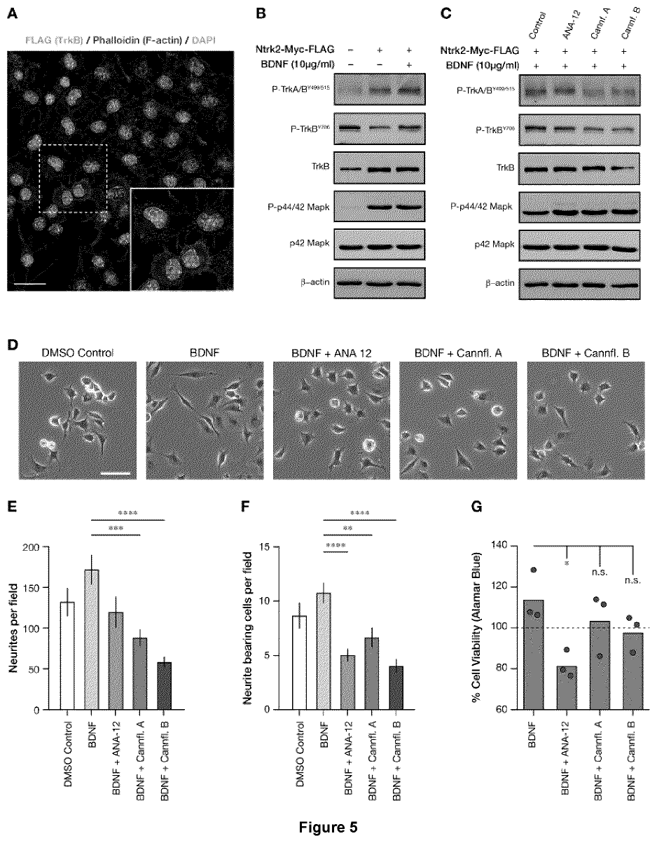

Our biochemical analyses with mouse primary cortical neurons suggest that cannflavins A and B have inhibitor activity towards TrkB receptors. In order to establish if this effect is sufficient to limit cellular processes under the control of BDNF signaling, we used neuroblastoma Neuro2a cells stably expressing Ntrk2-Myc-FLAG (FIG. 5A) and conducted a neurite outgrowth assay. As shown in FIG. 5B, Neuro2a cells have low expression of TrkB. Remarkably, though, cells that were transfected and selected for stably expressing the receptor became responsive to the exogenous application of BDNF, which is demonstrated by higher P-TrkB and P-MAPK levels (FIG. 5B). Moreover, pre-application of cannflavins (20 μM) with BDNF to the culture media for 6 hours reduced TrkB phosphorylation (FIG. 5C). Of note, we also used the known TrkB inhibitor ANA-12 as a positive control in this experiment. Interestingly, we did not observe a decrease in P-Mapk levels with treatment of ANA-12 and cannflavins like seen in cortical neurons. This could be due to differences in basal levels between the two cell lines, as the addition of BDNF to cortical neurons caused an increase in phosphorylation of the p-44 and p-42 subunits of MAPK, but no change in phosphorylation levels when BDNF was added to the Neuro2a cells. However, when analyzing the morphology of Neuro2a cells after 24-hour treatment (FIG. 5D), ANA-12 and cannflavins caused not only a decrease in viable cells (FIG. 5E), but also in the total number of neurites per field (FIG. 5F), and the number of cells with neurites twice the length of the cell (FIG. 5G). Altogether, our results suggest that cannflavins act on TrkB receptors, preventing BDNF activation of downstream signaling of the receptor.

### Cannflavins Reduce Viability, Migration and Invasion of GBM Cells

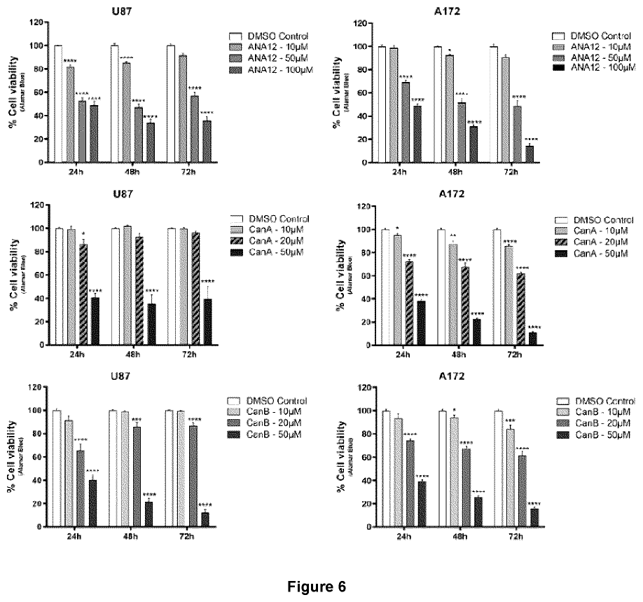

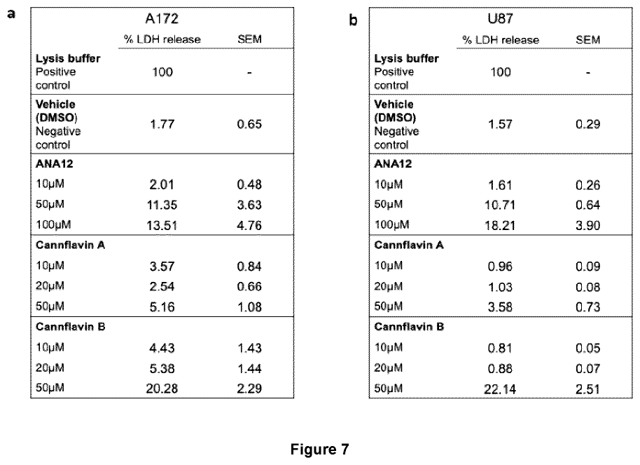

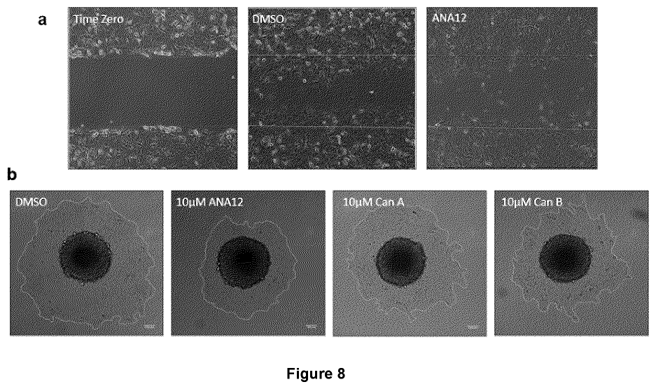

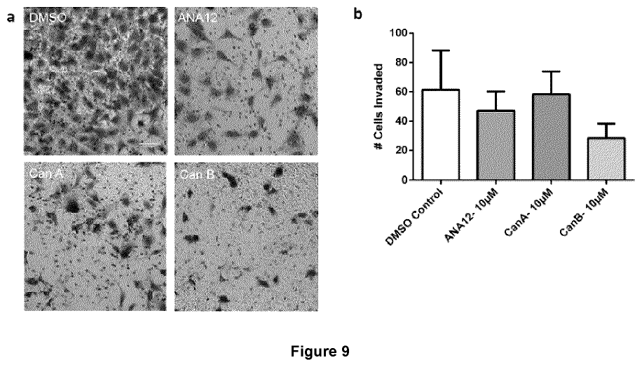

Addition of cannflavins into cultures of two types of glioblastoma cancerous cells (U87 and A172) showed a dose-dependent and time-dependent reduction of cell viability, similar to the observed selective TrkB inhibition by the inhibitor ANA-12, though both cannflavins were able to reduce viability at a lower concentration compared to ANA-12 (FIG. 6). Using a LDH cytotoxicity assay, it was determined that such reduction of viability by the cannflavins does not lead to cell necrosis in both glioblastoma cells tested (FIGS. 7A, 7B). Most importantly, through an in vitro technique used to assess the contribution of molecular and cellular mechanisms to cell migration (Scratch analysis, FIG. 8A), it was shown that cannflavins A and B were able to reduce the migration of the A172 and U87 GBM cells (FIG. 8B). In addition, results of a Boyden chamber assay showed that cannflavins A and B can reduce invasion of glioblastoma cells. When TrkB inhibitors were analyzed at low doses (10 μM) to prevent GBM cell invasion, both images of the assay (FIG. 9A) and quantification of invaded cells (FIG. 9B), showed that, despite the lack of statistical scores, cannflavin B tends to be more efficient in reducing cell invasion than cannflavin A and ANA-12. All these observations indicate that cannflavins present favourable anticancer activities.

### Discussion

Our study provides evidence for the antagonistic activity of cannflavins A and B, two small molecules derived from C. sativa, towards TrkB receptors. This result was unexpected since we had observed a potentiating effect of other flavonoids on BDNF-induced Arc expression in mouse primary cortical neurons. The addition of cannflavins in mouse cortical neurons prevents induction of Arc mRNA expression, suggesting that these molecules must act somewhere between BDNF-activation of TrkB and the transcription of Arc. Therefore, we investigated the effects of cannflavins on other downstream pathways of TrkB using biochemical analyses. Here, we detected a significant decrease in the activation of the Ras-Raf-MEK-MAPK and the PI3K/AKT/mTOR cascades. Furthermore, we demonstrated that cannflavins inhibited the BDNF-induced neurite outgrowth in TrkB overexpressed neuroblastoma cells.

Structural differences between Cannflavins A and B may explain the variance in antagonistic ability effect TrkB and downstream signaling. Notably, prenylation has been shown to affect the cytotoxicity of the flavonoids apigenin and liquiritigenin, where prenylated versions of these flavonoids increased the induction of apoptosis in hepatoma cells while maintaining antioxidative properties, compared to their non-prenylated counterparts (Watjen et al., 2007). The lack of prenylation in the control flavonoids EGCG, daidzein and genistein may explain how different flavonoids have divergent effects on TrkB signaling. Additionally, the differing degree of prenylation between cannflavin A and B may provide insight into biological activity. Cannflavin B contains one isoprene unit whereas cannflavin A contains an additional isoprene unit, making cannflavin A an overall larger molecule. We speculate that the reduced size of cannflavin B may facilitate a more structurally favorable binding affinity to the TrkB, which may explain why Cannflavin B typically shows a more cytotoxic response by antagonistically binding to TrkB, blunting downstream signaling responsible for proliferation and viability.

One member of this group that has attracted considerable attention over the years for its effect on BDNF/TrkB signaling is 7,8-DHF (Liu et al., 2016). Examination of the literature regarding 7,8-DHF, a molecule considered to have agonist TrkB activity, provides support to our observations (Liu et al., 2016). Cannflavins may not be optimal tools for neuroinflammation or pro-cognitive effects; however, cannflavins may prove to be valuable for other medical conditions where TrkB signaling is overactive or dysregulated. For instance, glioblastoma, breast tumors, lung cancer, pancreatic cancer are all reported to have irregular TrkB activity (Gupta et al., 2013). Notably, other small molecules of C. sativa have been explored in cancer research, for example, flavonoid derivatives increased apoptosis in pancreatic cancer (Moreau et al., 2019). Additionally, receptor tyrosine kinases represent a large family and whether cannflavins can block other known RTKs remains to be tested.

### REFERENCES

Antunes, L. C. M., Cartell, A., de Farias, C. B., Bakos, R. M., Roesler, R., Schwartsmann, G. (2019) Tropomyosin-related kinase receptor and neurotrophin expression in cutaneous melanoma is associated with a poor prognosis and decreased survival. Oncol (Basel) 97, 26-37.
Barbacid, M., Lamballe, F., Pulido, D., Klein, R. (1991) The trk family of tyrosine protein kinase receptors. Biochim. Biophys. Acta 1072, 115-127.
Barrett, M. L., Gordon, D., Evans, F. J. (1985) Isolation from Cannabis sativa L. of cannflavin—a novel inhibitor of prostaglandin production. Biochem. Pharmacol. 34, 2019-2024.
Barrett, M. L., Scutt, A. M., Evans, F. J. (1986) Cannflavin A and B, prenylated flavones from Cannabis sativa L. Experientia 42, 452-453.
Bramham, C. R., Alme, M. N., Bittins, M., Kuipers, S. D., Nair, R. R., Pai, B., Panja, D., Schubert, M., Soule, J., Tiron, A., Wibrand, K. (2010) The Arc of synaptic memory. Exp. Brain Res. 200, 125-140.
Cazorla, M., Prémont, J., Mann, A., Girard, N., Kellendonk, C., Rognan, D. (2011) Identification of a low-molecular weight TrkB antagonist with anxiolytic and antidepressant activity in mice. J Clin Invest. 121, 1846-1857.
Cocco, E., Scaltriti, M., Drilon, A. (2018) NTRK fusion-positive cancers and TRK inhibitor therapy. Nat. Rev. Clin. Oncol. 15, 731-747.
de Moraes, J. K., Wagner, V. P., Fonseca, F. P., do Amaral-Silva, G. K., de Farias, C. B., Pilar, E. F. S., Gregianin, A., Roesler, R., Vargas, P. A., Martins, M. D. (2019) Activation of BDNF/TrkB/Akt pathway is associated with aggressiveness and unfavorable survival in oral squamous cell carcinoma. Oral Dis 25, 1925-1936.
Ding, S., Zhuge, W., Hu, J., Yang, J., Wang, X., Wen, F., Wang, C., Zhuge, Q. (2018) Baicalin reverses the impairment of synaptogenesis induced by dopamine burden via the stimulation of GABAAR-TrkB interaction in minimal hepatic encephalopathy. Psychopharmacology 235, 1163-1178.
Eggers, C., Fujitani, M., Kato, R., Smid, S. (2019) Novel cannabis flavonoid, cannflavin A displays both a hormetic and neuroprotective profile against amyloid β-mediated neurotoxicity in PC12 cells: Comparison with geranylated flavonoids, mimulone and diplacone. Biochem. Pharmacol. 169, 113609.
El Zein, N., Badran, B. M., Sariban, E. (2007) The neuropeptide pituitary adenylate cyclase activating protein stimulates human monocytes by transactivation of the Trk/NGF pathway. (2007) Cell. Signal. 19, 152-162.
Flores-Sanchez, I. J., Verpoorte, R. (2008) Secondary metabolism in Cannabis. Phytochem. Rev. 7, 615-639.
Geiger, T. R., Peeper, D. S. (2005) The neurotrophic receptor TrkB in anoikis resistance and metastasis: a perspective. Cancer Res 65, 7033-7036.
Gundimeda, U., McNeill, T. H., Fan, T. K., Deng, R., Rayudu, D., Chen, Z., Cadenas, E., Gopalakrishna, R. (2014) Green tea catechins potentiate the neuritogenic action of brain-derived neurotrophic factor: role of 67-kDa laminin receptor and hydrogen peroxide. Biochem. Biophys. Res. Commun. 445, 218-224.
Gupta, V. K., You, Y., Gupta, V. B., Klistorner, A., Graham, S. L. (2013) TrkB receptor signalling: implications in neurodegenerative, psychiatric and proliferative disorders. Int. J. Mol. Sci. 14, 10122-10142.
Herrera-Hernández, M. G., Ramon, E., Lupala, C. S., Tena-Campos, M., Pérez, J. J., Garriga, P. (2017) Flavonoid allosteric modulation of mutated visual rhodopsin associated with retinitis pigmentosa. Sci. Rep. 7, 11167.
Huang, E. J., Reichardt, L. F. (2003) Trk receptors: Roles in neuronal signal transduction. Annu. Rev. Biochem. 72, 609-642.
Kedrov, A. V., Durymanov, M., Anokhin, K. V. (2019) The Arc gene: Retroviral heritage in cognitive functions. Neurosci. Biobehav. Rev. 99, 275-281.
Kim, M. S., Lee, W. S., Jin, W. (2016) TrkB promotes breast cancer metastasis via suppression of Runx3 and Keap1 expression. Mol Cells 39, 258-265.
Korb, E., Finkbeiner, S. (2011) Arc in synaptic plasticity: from gene to behavior. Trends Neurosci. 34, 591-598.
Kowiański, P., Lietzau, G., Czuba, E., Waśkow, M., Steliga, A., Moryś, J. (2018) BDNF: A key factor with multipotent impact on brain signaling and synaptic plasticity. Cell Mol. Neurobiol.
Kroeze, W. K., Sassano, M. F., Huang, X. P., Lansu, K., McCorvy, J. D., Giguère, P. M., Sciaky, N., Roth, B. L. (2015) PRESTO-Tango as an open-source resource for interrogation of the druggable human GPCRome. Nat. Struct. Mol. Biol. 22, 362-369.
Laetsch, T. W., Hong, D. S. (2021) Tropomyosin receptor kinase inhibitors for the treatment of TRK fusion cancer. Clin Cancer Res 27, 4974-4982.
Lalonde, J., Reis, S. A., Sivakumaran, S., Holland, C. S., Wesseling, H., Sauld, J. F., Alural, B., Zhao, W. N., Steen, J. A., Haggarty, S. J. (2017) Chemogenomic analysis reveals key role for lysine acetylation in regulating Arc stability. Nat. Commun. 8, 1659.
Lange, A. M., Lo, H. W. (2018) Inhibiting TRK proteins in clinical cancer therapy. Cancers (Basel) 10, 105.
Lawn, S., Krishna, N., Pisklakova, A., Qu, X., Fenstermacher, D. A., Fournier, M., Vrionis, F. D., Tran, N., Chan, J. A., Kenchappa, R. S., Forsyth, P. A. (2015) Neurotrophin signaling via TrkB and TrkC receptors promotes the growth of brain tumor-initiating cells. J Biol Chem. 290, 3814-3824.
Liu, C., Chan, C. B., Ye, K. (2016) 7,8-dihydroxyflavone, a small molecular TrkB agonist, is useful for treating various BDNF-implicated human disorders. Transl Neurodegener. 5, 2.
Lu, Y., Sun, G., Yang, F., Guan, Z., Zhang, Z., Zhao, J., Liu, Y., Chu, L., Pei, L. (2019) Baicalin regulates depression behavior in mice exposed to chronic mild stress via the Rac/LIMK/cofilin pathway. Biomed. Pharmacother. 116, 109054.
Meakin, S. O., MacDonald, J. I. S., Gryz, E. A., Kubu, C. J., Verdi, J. M. (1999) The signaling adapter FRS-2 competes with Shc for binding to the nerve growth factor receptor TrkA. J Biol Chem 274, 9861-9870.
Miknyoczki, S. J., Lang, D., Huang, L. Y., Klein-Szanto, A. J. P., Dionne, C. A., Ruggeri, B. A. (1999) Neurotrophins and Trk receptors in human pancreatic ductal adenocarcinoma expression patterns and effects on in vitro invasive behavior. Int J Cancer 81, 417-427.
Moreau, M., Ibeh, U., Decosmo, K., Bih, N., Yasmin-Karim, S., Toyang, N., Lowe, H., Ngwa, W. (2019) Flavonoid derivative of Cannabis demonstrates therapeutic potential in preclinical models of metastatic pancreatic cancer. Front. Oncol. 9, 660.
Ortega, J. T., Parmar, T., Jastrzebska, B. Flavonoids enhance rod opsin stability, folding, and self-association by directly binding to ligand-free opsin and modulating its conformation. (2019) J. Biol. Chem. 294, 8101-8122.
Pan, M., Han, H., Zhong, C., Geng, Q. (2012) Effects of genistein and daidzein on hippocampus neuronal cell proliferation and BDNF expression in H19-7 neural cell line. J. Nutr. Health Aging 16, 389-394.
Pulciani, S., Santos, E., Lauver, A. V., Long, L. K., Aaronson, S. A., Barbacid, M. (1982) Oncogenes in solid human tumours. Nature 300, 539-542.
Qian, X., Riccio, A., Zhang, Y. & Ginty, D. D. (1998) Identification and characterization of novel substrates of Trk receptors in developing neurons. Neuron 21, 1017-1029
Rajagopal, R., Chao, M. V. (2006) A role for Fyn in Trk receptor transactivation by G-protein-coupled receptor signaling. Mol. Cell Neurosci. 33, 36-46.
Rajagopal, R., Chen, Z. Y., Lee, F. S., Chao, M. V. (2004) Transactivation of Trk neurotrophin receptors by G-protein-coupled receptor ligands occurs on intracellular membranes. J. Neurosci. 24, 6650-6658.
Rea, K. A., Cararetto, J. A., AI-Abdul-Wahid, M. S., Sukumaran A., Geddes-McAlister J., Rothstein S. J., Akhtar T. A. (2019) Biosynthesis of cannflavins A and B from Cannabis sativa L. Phytochemistry 164, 162-171.
Shen, T., Cheng, X. S., Xia, C. F., Li, Q., Gao, Y., Pan, D. G., Zhang, X., Zhang, C., Li, Y. F. (2019) Erlotinib inhibits colon cancer metastasis through inactivation of TrkB-dependent ERK signaling pathway. J Cell Biochem. 120, 11248-11255.
Sinkevicius, K. W., Kriegel, C., Bellaria, K. J., Lee J., Lau A. N., Leeman K. T., Zhou P. C., Beede, A. M., Fillmore, C. M., Caswell, D., et al. (2014) Neurotrophin receptor TrkB promotes lung adenocarcinoma metastasis. Proc Natl Acad Sci USA 111, 10299-10304.
Solomon, J. P., Benayed, R., Hechtman, J. F., Ladanyi, M. (2019) Identifying patients with NTRK fusion cancer. Ann Oncol 30, viii16-viii22.
Thomaz A., Pinheiro K. D., Souza B. K., Gregianin L., Brunetto A. L., Brunetto A. T., de Farias C. B., Jaeger M. D., Ramaswamy V., Nor C., et al. (2019) Antitumor activities and cellular changes induced by TrkB inhibition in medulloblastoma. Front Pharmacol 10, 698.
Vaishnavi, A., Le, A. T., Doebele, R. C. (2015) TRKing down an old oncogene in a new era of targeted therapy. Cancer Discovery 5, 25-34.
Wang, Y., Long, P., Wang, Y., Ma, W. (2020) NTRK Fusions and TRK Inhibitors: Potential Targeted Therapies for Adult Glioblastoma. Front. Oncol. 10, 593578.
Wätjen, W., Weber, N., Lou, Y. J., Wang, Z. Q., Chovolou, Y., Kampkotter, A., Kahl, R., Proksch, P. (2007) Prenylation enhances cytotoxicity of apigenin and liquiritigenin in rat H4IIE hepatoma and C6 glioma cells. Food Chem. Toxicol. 145, 119-124.
Xu Y., Jiang W. G., Wang H. C., Martin T., Zeng Y. X., Zhang J., Qi Y. S. (2019) BDNF activates TrkB/PLC gamma 1 signaling pathway to promote proliferation and invasion of ovarian cancer cells through inhibition of apoptosis. Eur Rev Med Pharmacol Sci 23, 5093-5100.

1. A method for treating and/or preventing cancer, the method comprising administering to a subject in need thereof a therapeutically effective and a pharmaceutically acceptable amount of a cannabis-derived flavonoid.

2. The method of claim 1, wherein the cannabis-derived flavonoid inhibits Trk.

3. The method of claim 2, wherein the cannabis-derived flavonoid is cannflavin A and/or cannflavin B and/or cannflavin C.

4. The method of claim 1, wherein:
the cannabis-derived flavonoid decreases the activation of downstream pathways of TrkA, TrkB, and/or TrkC by disrupting signaling phosphorylation pathways of downstream kinases or proteins;
the cannabis-derived flavonoid reduces the viability of a cancerous cell in a dose and time-dependent manner;
the cannabis-derived flavonoid does not lead to cytotoxicity or cell necrosis;
the cannabis-derived flavonoid reduces cancerous cell migration;
the cannabis-derived flavonoid reduces cancerous cell invasion; and/or
the cannabis-derived flavonoid limits activation of TrkB by BDNF.

5. (canceled)

10. The method of claim 1, wherein the cancer is a RTK/Trk-associated cancer.

11. The method of claim 10, wherein the cancer comprises: brain cancers (e.g., glioblastoma multiforme, glioma, brain stem glioma), breast cancer, colorectal cancer, prostate cancer, pancreas cancer, ovarian cancer, lung cancer, bladder cancer, melanoma, thyroid cancer, head and neck cancers, uterine sarcoma, and/or neuroblastoma adrenocortical carcinoma.

12. (canceled)

13. The method of claim 1, comprising administration of an effective dose of a pharmaceutical composition comprising the at least one cannabis-derived flavonoid, and optionally at least one pharmaceutically acceptable carrier.

14. The method of claim 1, wherein the cannabis-derived flavonoid is administered separately, simultaneously, or sequentially with a Trk inhibitor, wherein the Trk inhibitor is optionally another cannabis-derived flavonoid selected from the group consisting of cannflavin A, cannflavin B, and cannflavin C, or combinations thereof.

15. The method of claim 1, further comprising administration of a flavonoid, comprising: chrysoeriol, isocannflavin B, canaflone (FBL-03G), hesperetin, acacetin, apigenin, luteolin, chrysin, quercetin, kaempferol, 8-prenyl-kaempferol, galangin, 6-prenylnaringenin, hesperetin, vitexin, wogonin, and/or delphinidin.

16. The method of claim 1, further comprising administration of an anticancer agent.

17. The method of claim 16, wherein the anticancer agent is a TrkA, TrkB, or TrkC inhibitor.

18.-25. (canceled)

26. A method for inhibiting Trk, the method comprising administering a cannabis-derived flavonoid.

27. The method of claim 26, wherein the cannabis-derived flavonoid is cannflavin A and/or cannflavin B and/or cannflavin C.

28. The method of claim 26, wherein the cannabis-derived flavonoid decreases the activation of downstream pathways of TrkA, TrkB, and/or TrkC by disrupting signaling phosphorylation pathways of downstream kinases or proteins.

29. The method of claim 26, for treating and/or preventing a RTK/Trk-associated cancer.

30.-31. (canceled)

32. The method of claim 26, wherein:
the cannabis-derived flavonoid reduces the viability of a cancerous cell in a dose and time-dependent manner;
the cannabis-derived flavonoid does not lead to cytotoxicity or cell necrosis;
the cannabis-derived flavonoid reduces cancerous cell migration;
the cannabis-derived flavonoid reduces cancerous cell invasion; and/or
the cannabis-derived flavonoid limits activation of TrkB by BDNF.

33.-44. (canceled)
